(19) United States
(12) Patent Application Publication
(10) Pub. No.: US 2025/0119285 A1
(43) Pub. Date: Apr. 10, 2025

(54) SYMMETRIC KEY EXCHANGE VIA TLS (TRANSPORT LAYER SECURITY) ON CAN (CONTROLLER AREA NETWORK) BUS INTERFACE

(71) Applicants: Baxter International Inc., Deerfield, IL (US); Baxter Healthcare SA, Glattpark (Opfikon) (CH)

(72) Inventors: Michael John Stelzner, Huntley, IL (US); David Schmidt, Gurnee, IL (US); Subbaiah Patibandla, Bangalore, Karnataka (IN)

(21) Appl. No.: 18/906,781

(22) Filed: Oct. 4, 2024

Related U.S. Application Data
(60) Provisional application No. 63/542,991, filed on Oct. 6, 2023.

(51) Int. Cl. H04L 9/08; H04L 9/32; H04L 12/40

(52) U.S. Cl. CPC H04L 9/0891; H04L 9/0825; H04L 9/3242; H04L 12/40104; H04L 2012/40215

(57) ABSTRACT

Symmetric key exchange via TLS on CAN bus interface for multiple devices. The key exchange is configured to elect a master PCA pump for a CAN bus network and prevent two PCA pumps from being master. Upon joining the CAN bus network PCA pumps exchange their MAC IDs with the PCA pumps in the CAN bus network. Once a bootstrap timeout ends, each PCA pump compares all MAC IDs to determine if it should advance to the voting stage. If the PCA pump has the lowest MAC ID, the PCA pump enters the voting stage. During the voting stage, the PCA pump sends master requests prompts to all the PCA pumps in the network. If the PCA pump's internal vote counter reaches a count of three the PCA pump becomes key master.

### CROSS-REFERENCE TO RELATED APPLICATIONS

The present application claims the benefit of U.S. Provisional Application No. 63/542,991, entitled SYMMETRIC KEY EXCHANGE VIA TLS (TRANSPORT LAYER SECURITY) ON CAN (CONTROLLER AREA NETWORK) BUS INTERFACE and filed Oct. 6, 2023, the contents of which are hereby incorporated by reference in their entirety.

### BACKGROUND

Patient Control Analgesia (PCA) pumps are commonly used for administering pain medication intravenously. In order to efficiently and secure transmit data with various other medical devices in a care setting, PCA pumps, and the other devices, must be able to communicate via a network. The preferred scheme is a Control Area Network (CAN) bus. A CAN bus is a half-duplex bus network. Moreover, in a medical setting, communications must be encrypted on a point to point manner and then be broadcasted. As part of existing information exchange schemes, there is use of Transport Layer Security (TLS), but this requires point to point connection between two devices. Relatedly, to exchange information over a CAN bus, keys are exchanged. Unfortunately, encrypted communication, point to point and broadcast, via CAN bus, when many devices are in a network, can present significant challenge for key exchange.

Currently, there are various key exchange methods via a CAN bus network, such as Diffie-Hellman. Unfortunately, existing key exchange methods make public key infrastructure (PKI) very “expensive” in terms of bandwidth utilization, memory use, and time. Specifically, key exchange via standard methods is very “costly” when used over a CAN bus communication. Existing key exchanges rely on significant amount of data communication between devices at a point to point level of communication. CAN bus has limited bandwidth for communication. Each device would need to do a key exchange with each other device. This would require ((n−1)*n)/2) key exchanges with the respective data for each exchange (amount of data for each exchange varies). The key exchange occurs during operation and there must be no interruption of device operation as the result of key exchange. Moreover, TLS requires a direct point to point connection between two devices. In a medical setting, PCA pump hubs need to utilize broadcast communication. Relying only on TLS would require each device to maintain ((n−1)*n)/2) tunnels, and repeat each packet (n−1) times. This would consume virtually all bandwidth on communication that is of no interest to any particular device. As such, for PCA pump hubs, current key exchange methodologies are not practical, effective methods.

A need accordingly exists for a key master for use by a multi-device system, where the key master maintains a rekey timer and updates keys as needed, and where each client connects to the key master to obtain the key.

### SUMMARY

Example systems, methods, and apparatus are disclosed herein for symmetric key exchange via TLS on CAN bus interface for multiple devices. The example systems, methods, and apparatus are configured to allow devices, such as PCA pumps, to attain master heartbeat status within a CAN bus network. Additionally, the example systems, methods, and apparatus are configured to allow devices in the CAN bus network to vote for a master device and for a new master device to be elected if the current master device is removed from the CAN bus network. The disclosed systems, methods, and apparatus minimize data usage on a CAN bus network by reducing the need to use excessive broadband communications because each device on the network does not need to be individually connected to each other device on the network. As such, the disclosed systems, methods, and apparatus increase network efficiency simplify communication between devices in a network, such as networks including PCA pumps.

In light of the disclosure herein, and without limiting the scope of the invention in any way, in a first aspect of the present disclosure, which may be combined with any other aspect listed herein unless specified otherwise, an infusion pump system including a plurality of infusion pumps, each of the infusion pumps including a MAC identifier stored in a memory device; a hub including a plurality of mechanical connectors, each mechanical configured to retain one of the plurality of infusion pumps when connected, a plurality of CAN interfaces, each CAN interface configured to communicatively couple to a respective infusion pump, and a CAN bus connected to each of the plurality of CAN interfaces; and each infusion pump is configured to: perform a first stage of a Bootstrap Timeout during which each of the plurality of PCA pumps creates a MAC ID table by broadcasting its MAC identifier and receiving broadcasts of MAC identifiers from the other infusion pumps, when the MAC identifier of the infusion pump is not the lowest MAC identifier within the MAC ID table, refrain from being designated as a potential key master, and when the MAC identifier of the infusion pump is the lowest MAC identifier within the MAC ID table, enter a second stage, and perform the second stage, in which the infusion pump transmits a master request message and its MAC identifier to the plurality of PCA pumps and increases its vote counter by one, concurrently subsequent potential key masters send their master request and MAC identifier to the infusion pump, and the infusion pump transmits a master reject to the subsequent potential key masters when their MAC identifiers have a higher value than the MAC identifier of the infusion pump and the infusion pump increases its vote count by one, and the infusion pump withdraws from voting and sets its counter to zero when at least one of the MAC identifiers of the other potential key masters is lower than the MAC identifier of the infusion pump. The infusion pump is designated as a key master when its vote counter reaches a value of three. The key master transmits a master reject to any additional master request message, the key master updates its heartbeat key status to master, and the key master starts a rekey timer.

In a second aspect of the present disclosure, which may be combined with any other aspect listed herein unless specified otherwise, the Bootstrap Timeout is 3 seconds.

In a third aspect of the present disclosure, which may be combined with any other aspect listed herein unless specified otherwise, the infusion pump withdraws from voting and sets its counter to zero when it receives a master reject.

In a fourth aspect of the present disclosure, which may be combined with any other aspect listed herein unless specified otherwise, the infusion pump increases its vote counter by one three seconds after it transmits the master request message and its MAC identifier to the plurality of PCA pumps.

In a fifth aspect of the present disclosure, which may be combined with any other aspect listed herein unless specified otherwise, the infusion pump includes at least one of a syringe pump, a PCA pump, a large volume pump, or a nutrition pump.

In a sixth aspect of the present disclosure, which may be combined with any other aspect listed herein unless specified otherwise, the infusion pumps further comprise a transceiver.

In a seventh aspect of the present disclosure, which may be combined with any other aspect listed herein unless specified otherwise, a networked system comprising: a plurality of devices, each of the devices including a MAC identifier stored in a memory device; a hub including: a plurality of mechanical connectors, each mechanical configured to retain one of the plurality of devices when connected, a plurality of CAN interfaces, each CAN interface configured to communicatively couple to a respective device, and a CAN bus connected to each of the plurality of CAN interfaces; and each device is configured to: perform a first stage of a Bootstrap Timeout during which each of the plurality of devices creates a MAC ID table by broadcasting its MAC identifier and receiving broadcasts of MAC identifiers from the other devices, when the MAC identifier of the device is not the lowest MAC identifier within the MAC ID table, refrain from being designated as a potential key master, and when the MAC identifier of the device is the lowest MAC identifier within the MAC ID table, enter a second stage, and perform the second stage, during which the device transmits a master request message and its MAC identifier to the plurality of devices and increases its vote counter by one, concurrently subsequent potential key masters send their master request and MAC identifier to the device, and the device transmits a master reject to the subsequent potential key masters when their MAC identifiers have a higher value than the MAC identifier of the infusion pump and the device increases its vote count by one, and the device withdraws from voting and sets its counter to zero when at least one of the MAC identifiers of the other potential key masters is lower than the MAC identifier of the device. The device is designated as a key master when its vote counter reaches a value of three. The key master transmits a master reject to any additional master request message, the key master updates its heartbeat key status to master, and the key master starts a rekey timer.

In an eighth aspect of the present disclosure, which may be combined with any other aspect listed herein unless specified otherwise, the Bootstrap Timeout is 3 seconds.

In a ninth aspect of the present disclosure, which may be combined with any other aspect listed herein unless specified otherwise, the device withdraws from voting and sets its counter to zero when it receives a master reject.

In a tenth aspect of the present disclosure, which may be combined with any other aspect listed herein unless specified otherwise, the device increases its vote counter by one three seconds after it transmits the master request message and its MAC identifier to the plurality of devices.

In an eleventh aspect of the present disclosure, which may be combined with any other aspect listed herein unless specified otherwise, the device is an infusion pump.

In a twelfth aspect of the present disclosure, which may be combined with any other aspect listed herein unless specified otherwise, the infusion pump includes at least one of a syringe pump, a PCA pump, a large volume pump, or a nutrition pump.

In a thirteenth aspect of the present disclosure, which may be combined with any other aspect listed herein unless specified otherwise, the infusion pumps further comprise a transceiver.

In a fourteenth aspect of the present disclosure, which may be combined with any other aspect listed herein unless specified otherwise, an infusion pump system comprising: a hub including: a plurality of mechanical connectors, each mechanical configured to retain one of the plurality of infusion pumps when connected, a plurality of CAN interfaces, each CAN interface configured to communicatively couple to a respective infusion pump, and a CAN bus connected to each of the plurality of CAN interfaces; and a plurality of infusion pumps. Each infusion pump includes a processor and a memory storing (i) a MAC identifier, and (ii) machine-readable instructions, which when executed, cause the processor of the infusion pump to: perform a first stage of a Bootstrap Timeout during which each of the plurality of PCA pumps creates a MAC ID table by broadcasting its MAC identifier and receiving broadcasts of MAC identifiers from the other infusion pumps, when the MAC identifier of the infusion pump is not the lowest MAC identifier within the MAC ID table, refrain from being designated as a potential key master, and when the MAC identifier of the infusion pump is the lowest MAC identifier within the MAC ID table, enter a second stage, and perform the second stage, during which the infusion pump transmits a master request message and its MAC identifier to the plurality of PCA pumps and increases its vote counter by one, concurrently subsequent potential key masters send their master request and MAC identifier to the infusion pump, and the infusion pump transmits a master reject to the subsequent potential key masters when their MAC identifiers have a higher value than the MAC identifier of the infusion pump and the infusion pump increases its vote count by one, and the infusion pump withdraws from voting and sets its counter to zero when at least one of the MAC identifiers of the other potential key masters is lower than the MAC identifier of the infusion pump. The infusion pump is designed as a key master when its vote counter reaches a value of three. The key master transmits a master reject to any additional master request message, the key master updates its heartbeat key status to master, and the key master starts a rekey timer.

In a fifteenth aspect of the present disclosure, which may be combined with any other aspect listed herein unless specified otherwise, the Bootstrap Timeout is 3 seconds.

In a sixteenth aspect of the present disclosure, which may be combined with any other aspect listed herein unless specified otherwise, the infusion pump withdraws from voting and sets its counter to zero when it receives a master reject.

In a seventeenth aspect of the present disclosure, which may be combined with any other aspect listed herein unless specified otherwise, the infusion pump increases its vote counter by one three seconds after it transmits the master request message and its MAC identifier to the plurality of PCA pumps.

In a eighteenth aspect of the present disclosure, which may be combined with any other aspect listed herein unless specified otherwise, the infusion pump includes at least one of a syringe pump, a PCA pump, a large volume pump, or a nutrition pump.

In a nineteenth aspect of the present disclosure, which may be combined with any other aspect listed herein unless specified otherwise, the infusion pumps further comprise a transceiver.

In a twentieth aspect of the present disclosure, which may be combined with any other aspect listed herein unless specified otherwise, the rekey timer is 1 minute, 5 minutes, 15 minutes, 30 minutes, 60 minutes, 90 minutes, or 120 minutes.

In a twenty-first aspect of the present disclosure, any of the structure, functionality, and alternatives disclosed in connection with any one or more of FIGS. 1 to 11 may be combined with any other structure, functionality, and alternatives disclosed in connection with any other one or more of FIGS. 1 to 11.

In light of the present disclosure and the above aspects, it is therefore an advantage of the present disclosure to remove the overhead of TLS tunnels.

It is another advantage of the present disclosure to eliminate the need for key exchange with each device in a network and the associated use of broadcast communication.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a system level diagram of symmetric key exchange via TLS on CAN bus interface for multiple PCA pumps within a hospital information system, according to an example embodiment of the present disclosure.

FIG. 2 is a perspective view of an example PCA pump comprising the Baxter® Novum pump, which may be included within the hospital system of FIG. 1, according to an example embodiment of the present disclosure.

FIG. 3 is an operations overview of symmetric key exchange via TLS on CAN bus interface for multiple PCA pumps within the hospital system of FIG. 1, according to an example embodiment of the present disclosure.

FIG. 4 is a flow diagram of symmetric key exchange via TLS on CAN bus interface for multiple PCA pumps within the hospital system of FIG. 1, according to an example embodiment of the present disclosure

FIG. 5 is an operations overview of stage 1 of symmetric key exchange via TLS on CAN bus interface for multiple PCA pumps within the hospital system of FIG. 1 when there is a single potential master, according to an example embodiment of the present disclosure.

FIG. 6 is an operations overview of stage 1 of symmetric key exchange via TLS on CAN bus interface for multiple PCA pumps within the hospital system of FIG. 1 when there is a single potential master and delays, according to an example embodiment of the present disclosure.

FIG. 7 is an operations overview of stage 1 of symmetric key exchange via TLS on CAN bus interface for multiple PCA pumps within the hospital system of FIG. 1 when there is more than one potential master due to an error, according to an example embodiment of the present disclosure.

FIG. 8 is an operations overview of stage 1 of symmetric key exchange via TLS on CAN bus interface for multiple PCA pumps within the hospital system of FIG. 1 when there are multiple potential masters, according to an example embodiment of the present disclosure.

FIG. 9 is an operations overview of stage 1 of symmetric key exchange via TLS on CAN bus interface for multiple PCA pumps within the hospital system of FIG. 1 when there is self-rejection by a PCA pump, according to an example embodiment of the present disclosure.

FIG. 10 is an operations overview of initial round of stage 2 of symmetric key exchange via TLS on CAN bus interface for multiple PCA pumps within the hospital system of FIG. 1, according to an example embodiment of the present disclosure.

FIG. 11 is an operations overview of final round of stage 2 of symmetric key exchange via TLS on CAN bus interface for multiple PCA pumps within the hospital system of FIG. 1, according to an example embodiment of the present disclosure.

### DETAILED DESCRIPTION

Methods, systems, and apparatus are disclosed herein for symmetric key exchange via TLS on CAN bus interface for multiple devices. The example methods, systems, and apparatus are configured to elect a master PCA pump for key exchanges in a CAN bus network with a plurality of PCA pumps. Additionally, the example methods, systems, and apparatus are configured to prevent any two PCA pumps being the key master. As such, the bandwidth for the network is not excessively taxed for symmetric key exchanges. When PCA pumps are connected to a hub, the PCA pumps join the CAN bus network. Upon joining the CAN bus network, during each PCA pump's bootstrap timeout, the PCA pumps use transceivers to exchange their MAC identifiers (IDs), from a memory, to all the existing PCA pumps in the CAN bus network. Once the bootstrap timeout ends, each PCA pump populates a MAC ID table in its memory and compares all the entries in the MAC ID table to determine if the PCA pump should advance to the voting stage. If the PCA pump has the lowest MAC ID within its MAC ID table, the PCA pump enters the voting stage. During the voting stage, the PCA pump sends master requests prompts to all the PCA pumps in the network. If the PCA pump's internal vote counter reaches a count of three, such that the PCA pump sends a master request without any master rejects from the other PCA pumps, the PCA pump becomes key master. Moreover, the method described herein is coded into the PCA pumps to be performed after the PCA pumps are connected to and receive an address from the hub. Thus, after the pumps are added to the hub, there is a handshake with the hub where the hub determines which pumps are located at which positons of pump modules. After the pumps are identified by the hub, they then determine which of the pumps is to be the key master. As such, there is no need for every PCA pump to exchange information individually with all other PCA pumps in the network, as the key master is elected to exchange the symmetric key. In turn, this removes the need to use already limited bandwidth in a CAN network for communication. Electing a key master is a solution because each PCA pump or device in the network will only need to connect to the key master to get a symmetric key for secure communication.

Reference is made herein to a memory. As disclosed herein, a memory refers to a device that holds electronic data and/or instructions for immediate use by a processor and/or device control system. The memory is able to receive and transmit data.

Reference is made herein to a processor. As disclosed herein, a processor refers to a device that executes instructions stored by the memory. The memory receives and transmits data.

Reference is made herein to a transceiver. As disclosed herein, a transceiver refers to a device that is a combination transmitter and receiver. The transceiver is able to receive and transmit data.

While the example methods, apparatus, and systems are disclosed herein as operating with medical devices, specifically PCA pumps, it should be appreciated that the methods, apparatus, and systems may be operable with other devices. For example, the methods, apparatus, and systems may provide for symmetric key exchange via TLS on CAN bus interface for applications in automotive, aviation, or agricultural devices.

### PCA Pump Environment Embodiment

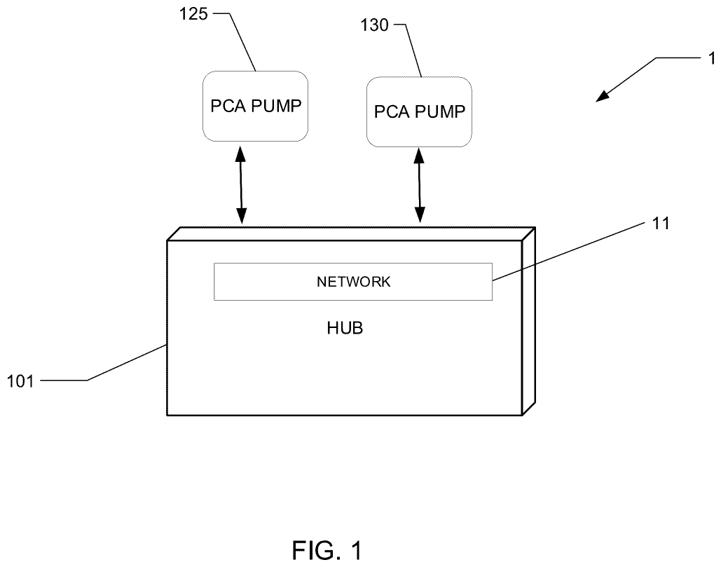

FIG. 1 is a system level diagram of symmetric key exchange via TLS on CAN bus interface for multiple PCA pumps within a hospital information system 1. The example system 1 includes multiple PCA pumps 125, 130 and a pump hub 101 with a CAN network (“network”) 11. The PCA pumps 125, 130 physically mount to the hub 101. The hub 101 includes a single medical device (PCA pump) docking apparatus and a connectivity stage. The connectivity stage connects the hub 101 to the CAN network 11. In an embodiment, the docking apparatus includes two shelves (not shown) for respectively receiving PCA pumps or other medical devices. In an embodiment, the shelves are configured to interchangeably accommodate different types of infusion pumps, such as syringe pumps, (Large Volume) LVP pumps, PCA pumps, etc. based on which type of pump is needed for a particular treatment. Further, depending on the number of PCA pumps needed, additional docking apparatuses may be added in a stacked configuration.

As such, the PCA pumps 125, 130 are physically and communicatively coupled to the hub 101 and, therefore, the CAN bus network 11. This means, the PCA pumps 125, 130 are part of the network 11 and, therefore, able to transmit and receive information with all devices in the network 11. While the present embodiment involves a CAN bus network, it must be noted that in an alternate embodiment, these connections may be wireless, such as via Bluetooth®, or wired, via a serial, Ethernet, or USB connection.

Furthermore, in alternate embodiments, other non-CAN local networks can be used, such as, but not limited to, Hart and Fieldbus networks.

In an alternate embodiment, the system 1 may also include a clinician device (not shown; e.g., a smartphone, tablet computer, laptop computer, workstation, etc.) such that a clinician can connect to the network 11 to monitor the PCA pumps 125, 130. Relatedly, in an alternate embodiment, additional devices such as, but not limited to, respirators, vitals monitors, or other medical devices are part of the system 1 and connect to the network 11.

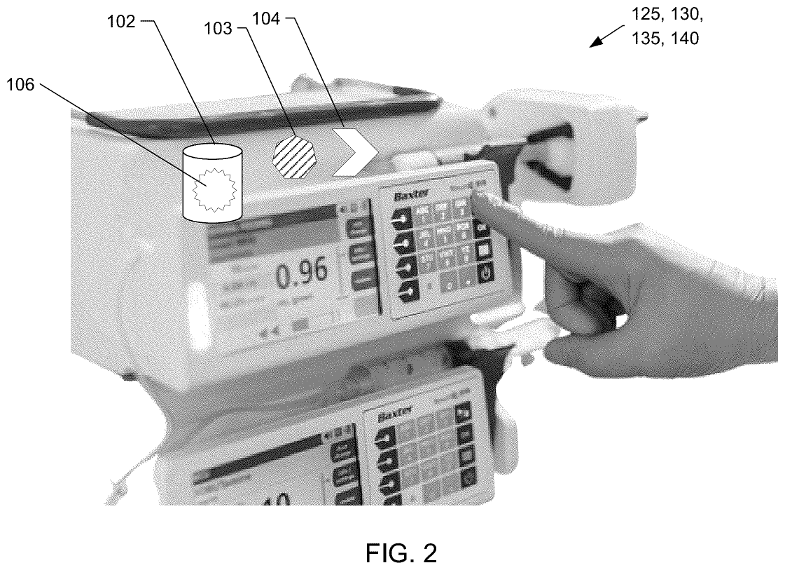

FIG. 2 is a perspective view of an example PCA pump 125, 130, 135, 140. The illustrated infusion pump 125, 130, 135, 140 is the Baxter® Novum IQ PCA pump. In this embodiment, the PCA pump 125, 130, 135, 140 includes a memory 102, a processor 103, and a transceiver 104. The memory 102 stores an unpopulated MAC ID table and a master request. The transceiver 104 receives a MAC IDs from other PCA pumps in the network. The processor 103 is configured to execute machine-readable instructions stored in the memory 102. Execution of the machine-readable instructions by the processor 103 causes the PCA pump 101 to perform the operations described herein.

Stage One & Stage Two Operations

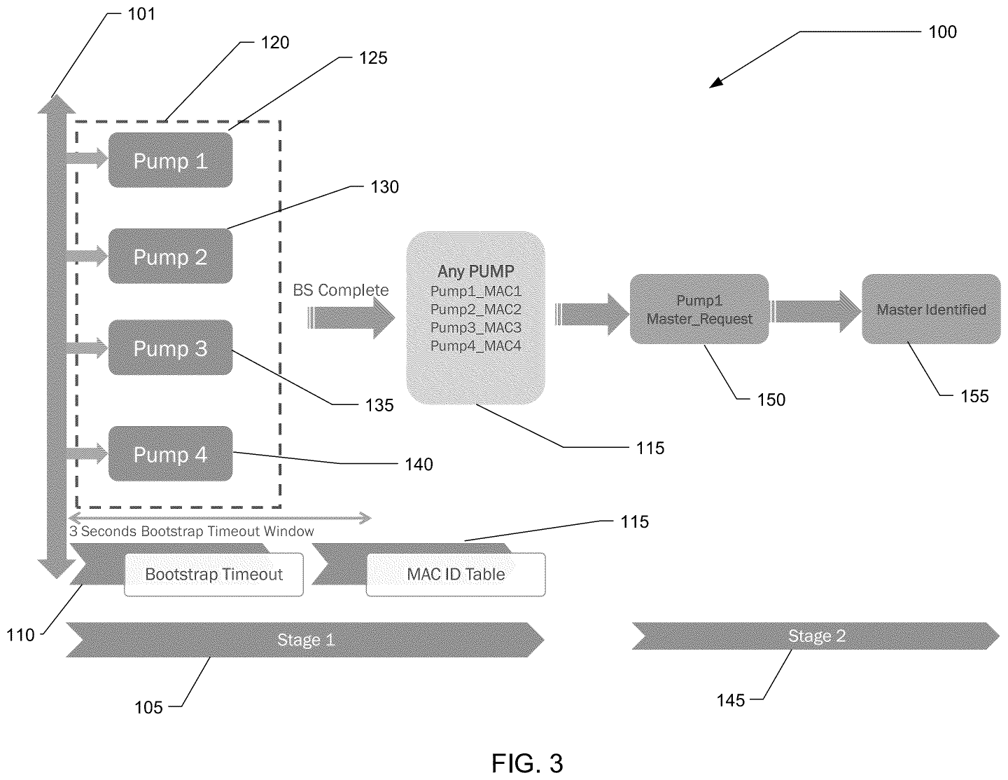

As noted previously, PCA pumps 125, 130, 135, 140 are connected to and communicate with the hub 101 (FIG. 3) and the network 11 (FIG. 1). Notably, each PCA pump 125, 130, 135, 140 has a unique MAC ID that is assigned during production.

FIG. 3 is an operations overview of a symmetric key exchange via TLS on CAN bus interface for multiple PCA pumps within the hospital system of FIG. 1. The symmetric key exchange involves a plurality of pumps 125, 130, 135, 140 which couple to a pump hub 101. Because the pump hub 101 includes a CAN network 11 (not shown in FIG. 3), the PCA pumps 125, 130, 135, 140 all connect to the CAN network 11 upon coupling to the pump hub 101.

The symmetric key exchange 100 comprises a stage one 105 of operations, followed by a stage two 145 of operations. Stage one 105 includes an initial bootstrap timeout 110, followed by a MAC identifier (MAC ID) table comparison 115. Stage two, also referred herein as a “voting stage,” 145 includes the exchange of master requests 150 followed by a master identification 155.

Beginning with stage one 105, specifically, with the bootstrap timeout 110, each PCA pump 125, 130, 135, 140 shares its MAC identifier with all PCA pumps 125, 130, 135, 140 in the network 101. In this embodiment, the bootstrap timeout 110 is three seconds long. In FIG. 3, all the PCA pumps 125, 130, 135, 140 are coupled to the 101, and therefore connected to the network 11, during the bootstrap timeout period 110. As a result, all PCA pumps 125, 130, 135, 140 exchange their MCA IDs with one another. At the conclusion of the bootstrap timeout 110, each device 125, 130, 135, 140 enters the MAC ID table build 115.

First, each PCA pump 125, 130, 135, 140 shares its MAC identifier with all PCA pumps in the network. Relatedly, each PCA pump 125, 130, 135, 140, receives the MAC identifiers for all other PCA pumps 125, 130, 135, 140 through its transceiver 104. The PCA pump 125, 130, 135, 140 stores all MAC IDs in a MAC table in its memory 102. Next, the processor 103 in each PCA pump 125, 130, 135, 140 retrieves the MAC ID table, and instructions to compare all MAC IDs, from the memory 102. The processor 103 in each PCA pump 125, 130, 135, 140 compares all the MAC IDs in each PCA pump's MAC table as further explained below.

Next, the PCA pumps 125, 130, 135, 140 enter stage two 145, also known as the voting stage. During the voting stage 145, each PCA pump 125, 130, 135, 140 At the conclusion of the MAC ID table comparison 115 each PCA pump 125, 130, 135, 140 determines if it should send a master request respective PCA pump 125, 130, 135, 140. During the voting stage 145, each potential key master (PCA pump 125 in this example) sends a master request 150 to all other PCA pumps 130, 135, 140. In accordance with the operations further explained below, the potential key master (125 in this example) can be identified as key master 155. Once a potential key master 125 is identified as key master 155, the key master updates its heartbeat key status to “master,” automatically rejects any master requests received immediately with a master reject, and starts a rekey timer. The rekey timer can range from seconds, to minutes, or hours. It should be noted that if the key master is removed or disappears from the network, the previously explained operations are repeated in the system to elect a new master.

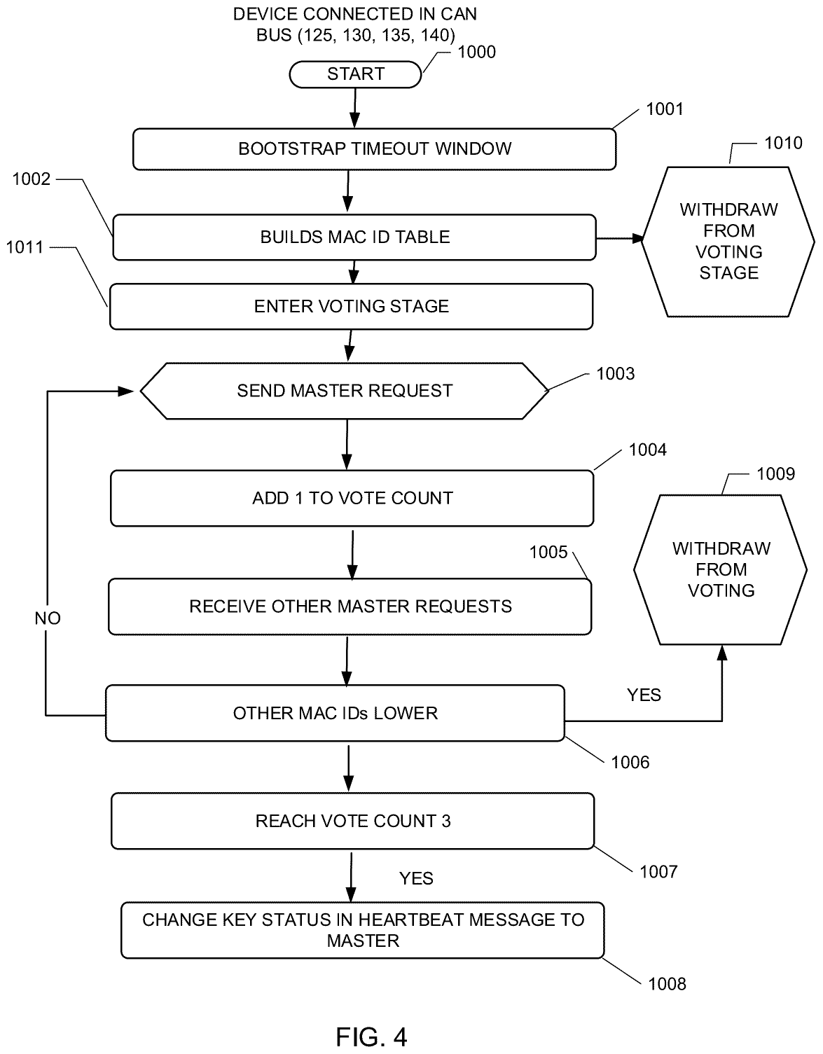

FIG. 4 is a flow diagram of symmetric key exchange via TLS on CAN bus interface for multiple PCA pumps within the hospital system of FIG. 1. As seen in the figure, first a device is connected to a CAN bus network 1000. In this situation, the device is a PCA pump 125, 130, 135, 140 as seen in FIG. 3 and the connection to the BUS can network happens when the PCA pump 125, 130, 135, 140 couples to the hub 101. During the bootstrap timeout window, each PCA pump transmits its MAC ID to all the PCA pumps connected to the network 1001. Next, each PCA pump populates a MAC ID table with the MAC IDs it received from other PCA pumps.

Each PCA pump then compares all the MAC IDs in the MAC ID table to determine if the respective PCA pump will enter the next stage 1002. If the respective PCA pump has a MAC ID value that is higher than the other MAC ID values in the MAC ID table, then the respective PCA pump withdraws from the voting stage 1010. However, if the respective PCA pump has a MAC ID value that is lower than the other MAC ID values in the MAC ID table, then the respective PCA pump enters the voting stage 1011. Specifically, the respective PCA pump sends a master request to the other PCA pumps connected the hub 1003. The master request includes both a request to be key master and the respective PCA pump's MAC ID. Concurrently with sending the master request, the respective PCA increments its internal counter to one 1004.

Also concurrently, the respective PCA pump receives master requests from other PCA pumps 1005. Upon receiving master requests from other PCA pumps, the respective pump compares the MAC IDs of the other PCA pumps 1006. If the other PCA pumps have a lower MAC ID, then the respective PCA pump withdraws from voting 1009 and sets its internal vote counter to zero.

By contrast, if the other PCA pumps have a higher MAC ID, then the respective PCA pump sends another master request and repeats the process described about until the respective PCA pump's internal counter reaches three 1007. Once the respective PCA pump's internal counter reaches three, the respective PCA pump becomes master 1008. Once the respective PCA pump becomes master, it responds to any master requests with a master reject that prompts the senders of those master requests to withdraw from voting and set their respective internal counters to zero. Also, once the respective PCA pump becomes master, it updates its heartbeat key status to “master.” Finally, once the respective PCA pump becomes master, it starts a rekey timer.

### Stage One: Single Potential Master

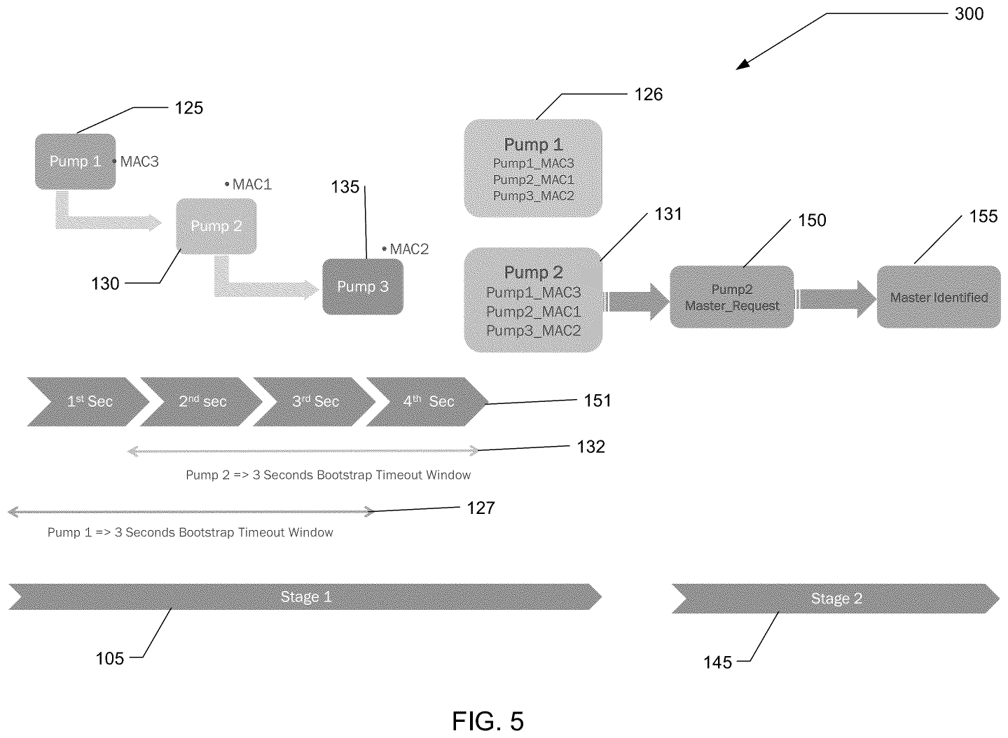

FIG. 5 is an operations overview of stage 1 of symmetric key exchange via TLS on CAN bus interface for multiple PCA pumps within the hospital system of FIG. 1 when there is a single potential master 300. In this example, there are three pumps: pump one 125, pump two 130, and pump three 135. In accordance with the operations previously described, stage one 105 begins when pump one 125 is the first to join the network at second one, at which point pump one's 3-second bootstrap timeout 127 begins. Pump two 130 joins the network at second two, at which point pump two's 3-second bootstrap timeout 132 begins. Pump three 135 joins the network at second three. As such, pump one 125 first populates its MAC ID table 126 at second three and determines, since pump one's MAC ID is not the lowest of all the MAC IDs in its table, not to enter the voting stage 145. By contrast, pump two 130 next populates its MAC ID table 131 at second four and determines, since pump two's MAC ID is the lowest of all the MAC IDs in its table, to enter the voting stage 145.

As a result, in the voting stage 145, pump two 130 sends a master request 150 and increased its internal vote counter as previously described. Pump two 130 continues to send a master request 150 and increase its internal counter until pump two's internal counter reaches three and, as such, pump two 130 becomes master 155.

### Stage One: Single Potential Master with Delays

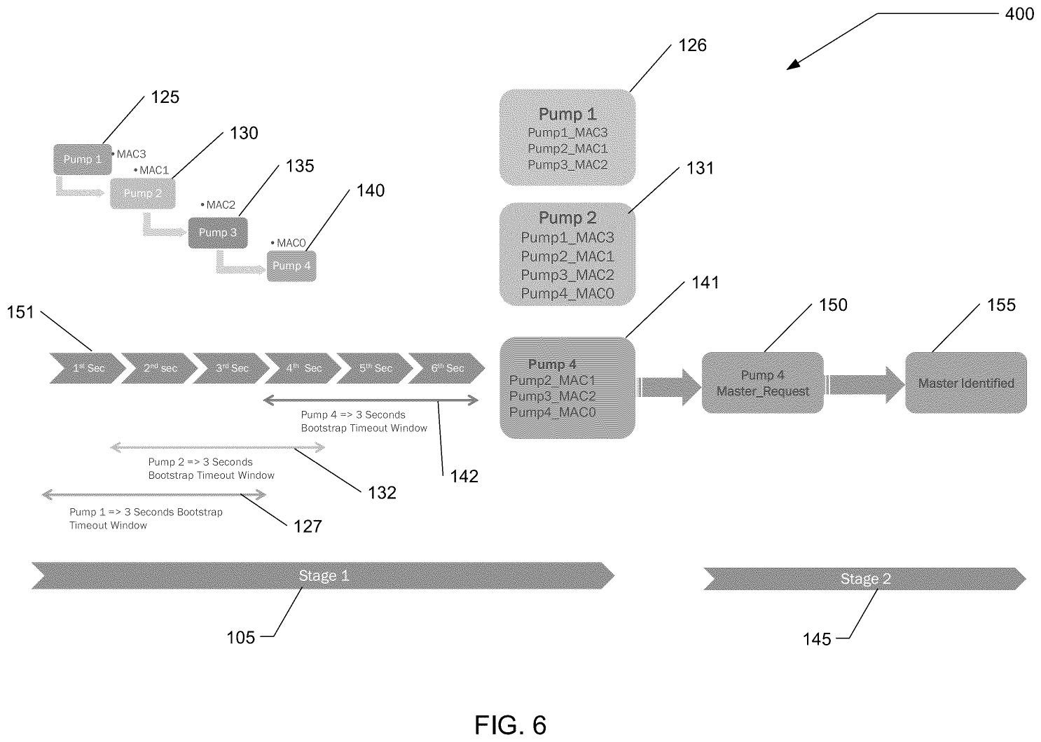

FIG. 6 is an operations overview of stage 1 of symmetric key exchange via TLS on CAN bus interface for multiple PCA pumps within the hospital system of FIG. 1 when there is a single potential master and delays 400. In this example, there are four pumps: pump one 125, pump two 130, pump three 135, and pump four 140. Stage one 105 begins when pump one 125 is the first to join the network at second one, at which point pump one's 3-second bootstrap timeout 127 begins. Pump two 130 joins the network at second two, at which point pump two's 3-second bootstrap timeout 132 begins. Pump three 135 joins the network at second three. Pump four 140 joins at second four 142.

As such, pump one 125 first populates its MAC ID table 126 at second three and determines, since pump one's MAC ID is not the lowest of all the MAC IDs in its table, not to enter the voting stage 145. Pump two 130 next populates its MAC ID table 131 at second four, thereby including pump four's MAC ID. Pump two 130 therefore determines that pump two's MAC ID is not the lowest in its table, and determines, not to enter the voting stage 145. However, pump four 140 enters the voting stage 145 because pump four 140 has the lowest MAC ID in its MAC ID table 141.

Similar to the previous example, in the voting stage 145, pump four 140 sends a master request 150 and increases its internal vote counter. Pump four 140 continues to send a master request 150 and increase its internal counter until pump four's internal counter reaches three and, as such, pump four 140 becomes master 155.

### Stage One: Multiple Potential Masters

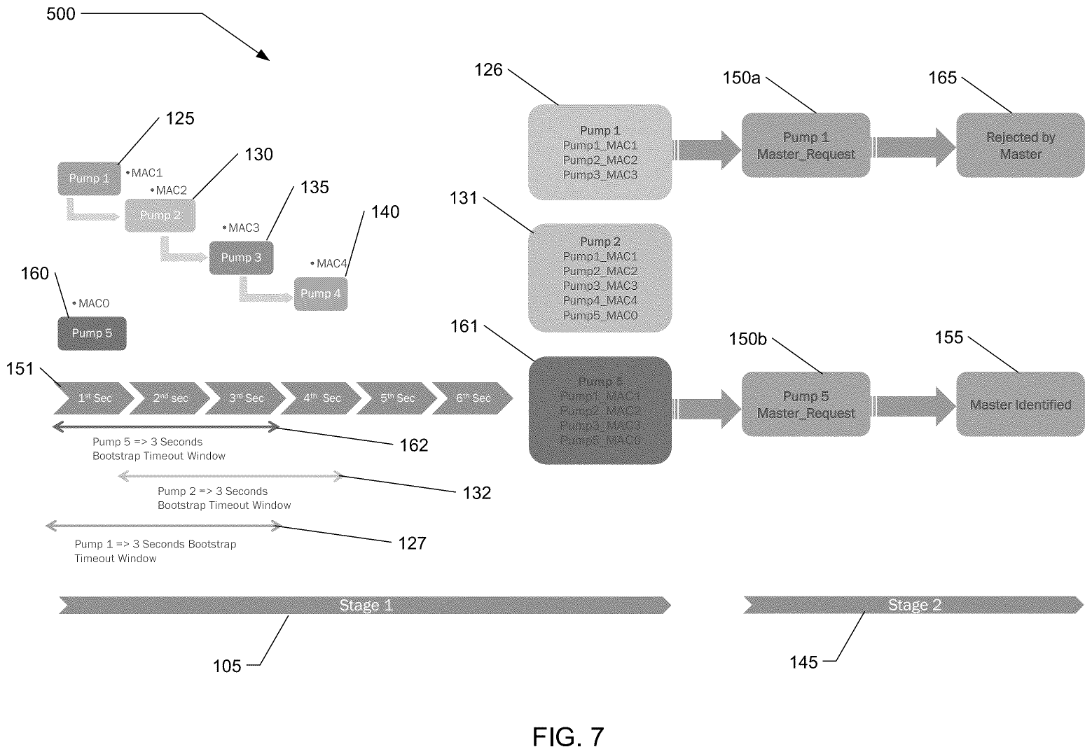

FIG. 7 is an operations overview of stage 1 of symmetric key exchange via TLS on CAN bus interface for multiple PCA pumps within the hospital system of FIG. 1 when there is more than one potential master due to an error 500. In this example, there are five pumps: pump one 125, pump two 130, pump three 135, pump four 140, and pump five 160. Stage one 105 begins when pump one 125 and pump five 160 join the network at second one, at which point pump one's 3-second bootstrap timeout 127 and pump five's 3-second bootstrap timeout 162 begin. Pump two 130 joins the network at second two, at which point pump two's 3-second bootstrap timeout 132 begins. Pump three 135 joins the network at second three. Pump four 140 joins at second four.

For illustrative purposes, in this example, pump one 125 populates its MAC ID table 126 with the MAC IDs for pump two and three 130, 135, but not pump five 160. As such, pump one 125, has lowest of all the MAC IDs in its table, and enters the voting stage 145. Similarly, pump five 160 populates its MAC ID table 161 with MAC IDs from pump one, two, and three 125, 130, 135. Therefore, pump five 160, has lowest of all the MAC IDs in its table, and also enters the voting stage 145. Pump two 130 next populates its MAC ID table 131 and determines, not to enter the voting stage 145.

Unlike previous examples, in this voting stage 145, pump one 125 sends a master request 150a and pump five 160 sends a master request 150b. In this example, when pump five 160 receives a master request 150a, it compares the MAC ID number included in the master request 150a, which is pump one's MAC ID, and pump five 160 determines that its own MAC ID is lower than pump one's MAC ID. As a result, pump five 160 sends a master reject 165, which prompts pump one 125 to withdraw from voting. In turn similar to the previous examples, in the voting stage 145, pump five continues to send a master request 150 and increase its internal counter until pump five's internal counter reaches three and, as such, pump five 160 becomes master 155.

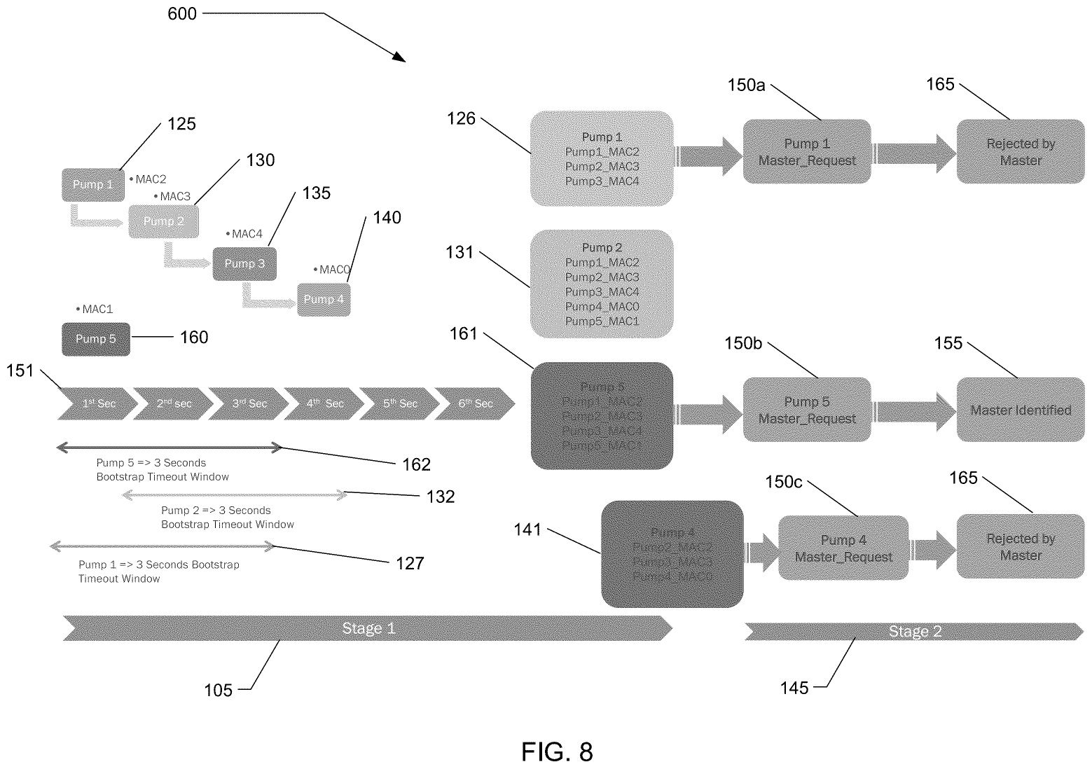

FIG. 8 shows a similar example involving multiple potential masters. FIG. 8 is an operations overview of stage 1 of symmetric key exchange via TLS on CAN bus interface for multiple PCA pumps within the hospital system of FIG. 1 when there are multiple potential masters 600. In this example, there are five pumps: pump one 125, pump two 130, pump three 135, pump four 140, and pump five 160. Stage one 105 begins when pump one 125 and pump five 160 join the network at second one, at which point pump one's 3-second bootstrap timeout 127 and pump five's 3-second bootstrap timeout 162 begin. Pump two 130 joins the network at second two, at which point pump two's 3-second bootstrap timeout 132 begins. Pump three 135 joins the network at second three. Pump four 140 joins at second four.

For illustrative purposes, in this example, pump one 125 populates its MAC ID table 126 with the MAC IDs for pump two and three 130, 135, but not pump five 160. As such, pump one 125, has lowest of all the MAC IDs in its table, and enters the voting stage 145. Similarly, pump five 160 populates its MAC ID table 161 with MAC IDs from pump one, two, and three 125, 130, 135. Therefore, pump five 160, has lowest of all the MAC IDs in its table, and also enters the voting stage 145. Pump two 130 next populates its MAC ID table 131 and determines, not to enter the voting stage 145. Pump four 140 also enters the voting stage because its MAC ID table 141 includes the MAC IDs for pump two 130 and pump three 135, and pump four's MAC ID is the lowest of those three MAC IDs.

In this voting stage 145, pump one 125 sends a master request 150a and pump five 160 sends a master request 150b. In this example, when pump five 160 receives a master request 150a, it compares the MAC ID number included in the master request 150a, which is pump one's MAC ID, and pump five 160 determines that its own MAC ID is lower than pump one's MAC ID. As a result, pump five 160 sends a master reject 165, which prompts pump one 125 to withdraw from voting. In turn similar to the previous examples, in the voting stage 145, pump five continues to send a master request 150 and increase its internal counter until pump five's internal counter reaches three and, as such, pump five 160 becomes master 155.

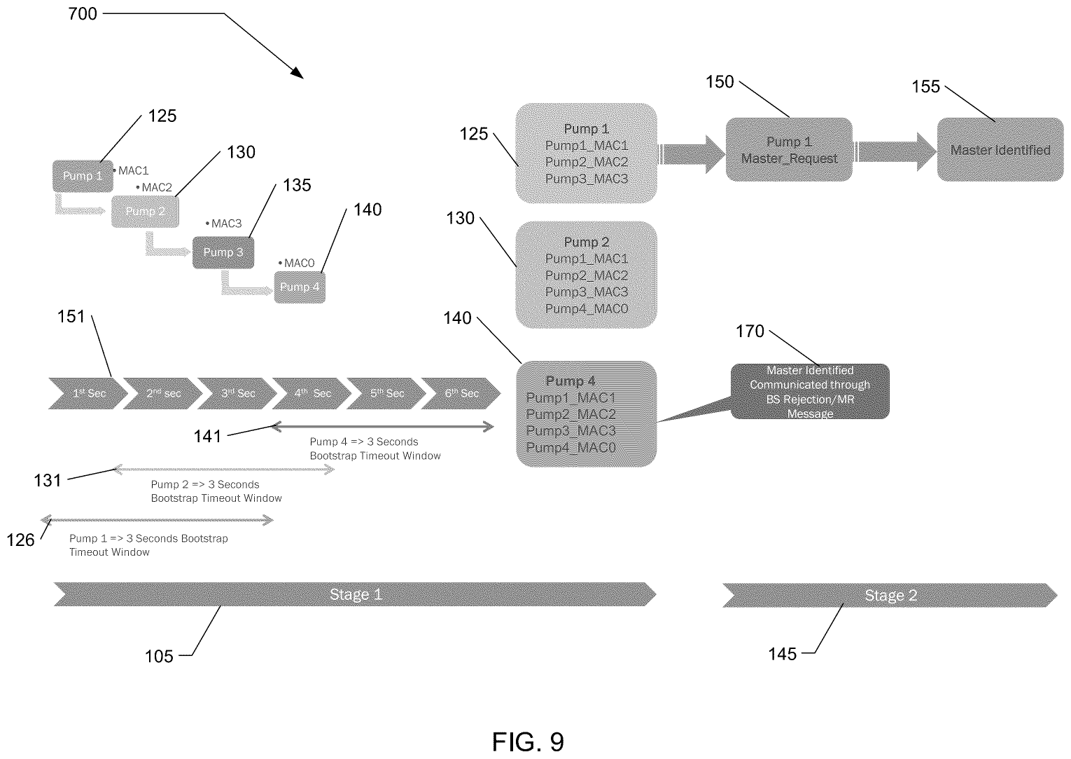

Pump four 140 sends a master request 150c, but pump five 160, which is already master, immediately responds by sending a master reject 165, which prompts pump four 140 to withdraw from voting. As such, FIG. 8 clearly illustrates an example where a pump with the overall lowest MAC ID (pump four 160) sends a master request after a master has already been identified, and the pump (pump four 160) is therefore rejected from being master. This is further seen in FIG. 9, which shows a similar situation, and further illustrates that once a master is identified by the system, a basic status rejection and master reject message 170 is communicated to all pumps (130, 140) that are not the key master (125 in this example).

### Stage Two

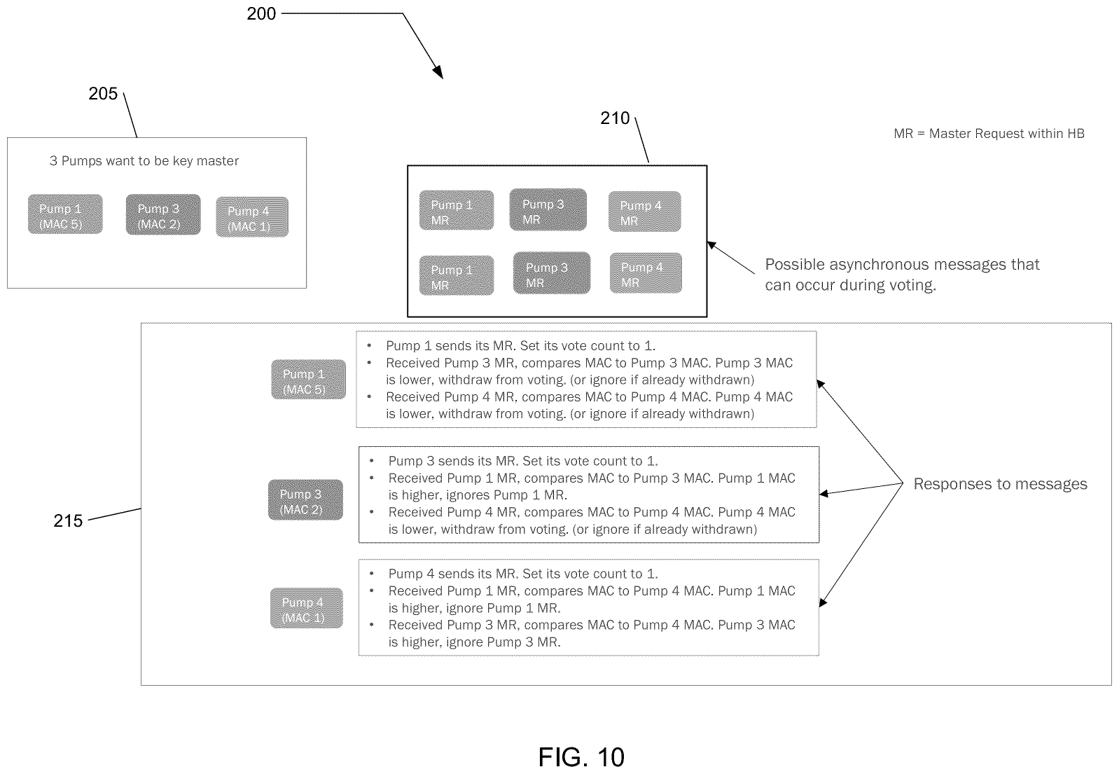

FIG. 10 is an operations overview of initial round of stage 2 of symmetric key exchange via TLS on CAN bus interface for multiple PCA pumps within the hospital system of FIG. 1 200. As seen in the image, once there are multiple pumps seeking to be master 205, there are multiple potential asynchronous message combinations 210 that can occur during stage two, the voting stage. Box 215 details various master requests sent between pumps, the associated comparisons of MAC IDs in each pump's respective MAC ID tables, and the associated updates to internal vote counters for each respective pump. Notably, all the messaging schemes shown in box 215 have been explained above with relation to FIGS. 3 to 9.

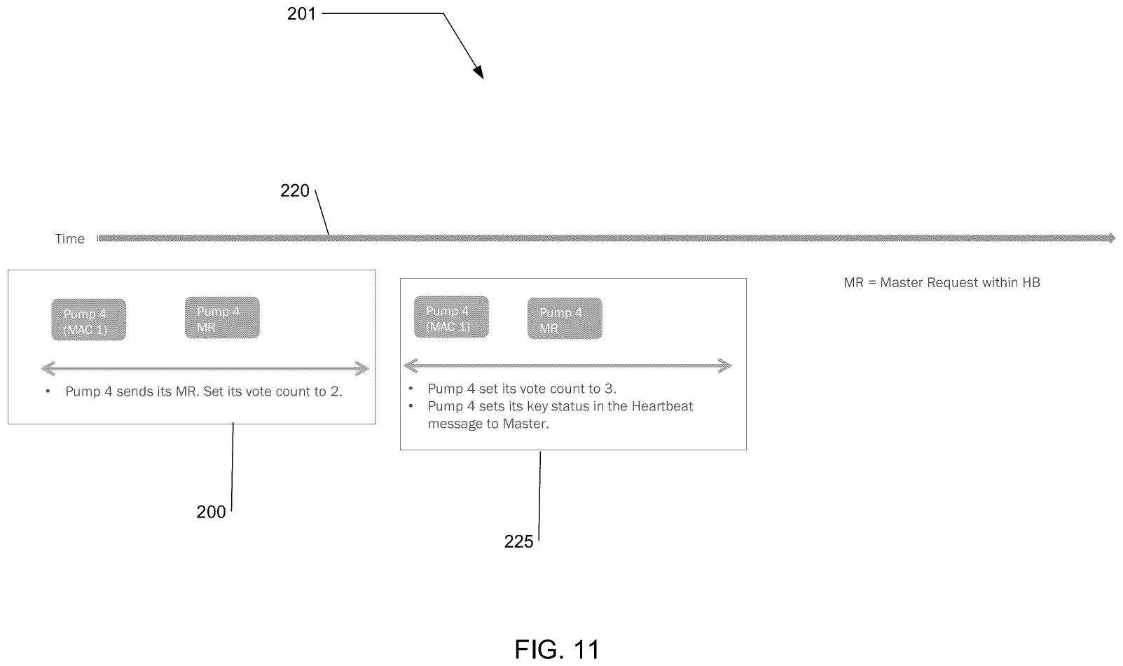

FIG. 11 is an operations overview of final round of stage 2 of symmetric key exchange via TLS on CAN bus interface for multiple PCA pumps within the hospital system of FIG. 1 201. As seen in the timeline overview 220, the initial round 200 of stage 2 concludes and immediately thereafter, the final round 225 of stage 2 takes place. As seen in box 225, and as previously explained, during the final round, a potential key master's internal vote counter reaches three and, as a result, the potential key master becomes the key master and sets its key status in the heartbeat message to master.

The operations described above allow symmetric key exchange in a multiple device network dynamically and without excessive bandwidth utilization.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. An infusion pump system comprising:
a plurality of infusion pumps, each of the infusion pumps including a MAC identifier stored in a memory device;
a hub including:
a plurality of mechanical connectors, each mechanical configured to retain one of the plurality of infusion pumps when connected,
a plurality of CAN interfaces, each CAN interface configured to communicatively couple to a respective infusion pump, and
a CAN bus connected to each of the plurality of CAN interfaces; and
wherein each infusion pump is configured to:
perform a first stage of a Bootstrap Timeout during which each of the plurality of PCA pumps creates a MAC ID table by broadcasting its MAC identifier and receiving broadcasts of MAC identifiers from the other infusion pumps,
when the MAC identifier of the infusion pump is not the lowest MAC identifier within the MAC ID table, refrain from being designated as a potential key master, and
when the MAC identifier of the infusion pump is the lowest MAC identifier within the MAC ID table, enter a second stage, and
perform the second stage, wherein the infusion pump transmits a master request message and its MAC identifier to the plurality of PCA pumps and increases its vote counter by one, concurrently subsequent potential key masters send their master request and MAC identifier to the infusion pump, and the infusion pump transmits a master reject to the subsequent potential key masters when their MAC identifiers have a higher value than the MAC identifier of the infusion pump and the infusion pump increases its vote count by one, and the infusion pump withdraws from voting and sets its counter to zero when at least one of the MAC identifiers of the other potential key masters is lower than the MAC identifier of the infusion pump,
wherein the infusion pump is designated as a key master when its vote counter reaches a value of three, and
wherein the key master transmits a master reject to any additional master request message and the key master updates its heartbeat key status to master, and the key master starts a rekey timer.

2. The system of claim 1, wherein the Bootstrap Timeout is 3 seconds.

3. The system of claim 1, wherein the infusion pump withdraws from voting and sets its counter to zero when it receives a master reject.

4. The system of claim 1, wherein the infusion pump increases its vote counter by one three seconds after it transmits the master request message and its MAC identifier to the plurality of PCA pumps.

5. The system of claim 1, wherein the infusion pump includes at least one of a syringe pump, a PCA pump, a large volume pump, or a nutrition pump.

6. The system of claim 1, wherein the infusion pumps further comprise a transceiver.

7. A networked system comprising:
a plurality of devices, each of the devices including a MAC identifier stored in a memory device;
a hub including:
a plurality of mechanical connectors, each mechanical configured to retain one of the plurality of devices when connected,
a plurality of CAN interfaces, each CAN interface configured to communicatively couple to a respective device, and
a CAN bus connected to each of the plurality of CAN interfaces; and
wherein each device is configured to:
perform a first stage of a Bootstrap Timeout during which
each of the plurality of devices creates a MAC ID table by broadcasting its MAC identifier and receiving broadcasts of MAC identifiers from the other devices,
when the MAC identifier of the device is not the lowest MAC identifier within the MAC ID table, refrain from being designated as a potential key master, and
when the MAC identifier of the device is the lowest MAC identifier within the MAC ID table, enter a second stage, and
perform the second stage, wherein the device transmits a master request message and its MAC identifier to the plurality of devices and increases its vote counter by one, concurrently subsequent potential key masters send their master request and MAC identifier to the device, and the device transmits a master reject to the subsequent potential key masters when their MAC identifiers have a higher value than the MAC identifier of the infusion pump and the device increases its vote count by one, and the device withdraws from voting and sets its counter to zero when at least one of the MAC identifiers of the other potential key masters is lower than the MAC identifier of the device,
wherein the device is designated as a key master when its vote counter reaches a value of three, and
wherein the key master transmits a master reject to any additional master request message and the key master updates its heartbeat key status to master, and the key master starts a rekey timer.

8. The system of claim 7, wherein the Bootstrap Timeout is 3 seconds.

9. The system of claim 7, wherein the device withdraws from voting and sets its counter to zero when it receives a master reject.

10. The system of claim 7, wherein the device increases its vote counter by one three seconds after it transmits the master request message and its MAC identifier to the plurality of devices.

11. The system of claim 7, wherein the device is an infusion pump.

12. The system of claim 11, wherein the infusion pump includes at least one of a syringe pump, a PCA pump, a large volume pump, or a nutrition pump.

13. The system of claim 7, wherein the infusion pumps further comprise a transceiver.

14. An infusion pump system comprising:
a hub including:
a plurality of mechanical connectors, each mechanical configured to retain one of the plurality of infusion pumps when connected,
a plurality of CAN interfaces, each CAN interface configured to communicatively couple to a respective infusion pump, and
a CAN bus connected to each of the plurality of CAN interfaces; and
a plurality of infusion pumps, each infusion pump including a processor and a memory storing (i) a MAC identifier, and (ii) machine-readable instructions, which when executed, cause the processor of the infusion pump to:
perform a first stage of a Bootstrap Timeout during which
each of the plurality of PCA pumps creates a MAC ID table by broadcasting its MAC identifier and receiving broadcasts of MAC identifiers from the other infusion pumps,
when the MAC identifier of the infusion pump is not the lowest MAC identifier within the MAC ID table, refrain from being designated as a potential key master, and
when the MAC identifier of the infusion pump is the lowest MAC identifier within the MAC ID table, enter a second stage, and
perform the second stage, wherein the infusion pump transmits a master request message and its MAC identifier to the plurality of PCA pumps and increases its vote counter by one, concurrently subsequent potential key masters send their master request and MAC identifier to the infusion pump, and the infusion pump transmits a master reject to the subsequent potential key masters when their MAC identifiers have a higher value than the MAC identifier of the infusion pump and the infusion pump increases its vote count by one, and the infusion pump withdraws from voting and sets its counter to zero when at least one of the MAC identifiers of the other potential key masters is lower than the MAC identifier of the infusion pump,
wherein the infusion pump is designated as a key master when its vote counter reaches a value of three, and
wherein the key master transmits a master reject to any additional master request message and the key master updates its heartbeat key status to master, and the key master starts a rekey timer.

15. The system of claim 14, wherein the Bootstrap Timeout is 3 seconds.

16. The system of claim 14, wherein the infusion pump withdraws from voting and sets its counter to zero when it receives a master reject.

17. The system of claim 14, wherein the infusion pump increases its vote counter by one three seconds after it transmits the master request message and its MAC identifier to the plurality of PCA pumps.

18. The system of claim 14, wherein the infusion pump includes at least one of a syringe pump, a PCA pump, a large volume pump, or a nutrition pump.

19. The system of claim 14, wherein the infusion pumps further comprise a transceiver.

20. The system of claim 14, wherein the rekey timer is 1 minute, 5 minutes, 15 minutes, 30 minutes, 60 minutes, 90 minutes, or 120 minutes.
